(19) United States
(12) Patent Application Publication
(10) Pub. No.: US 2008/0058676 A1
(43) Pub. Date: Mar. 6, 2008

(54) RETRACTABLE SEGMENTED BIO-MOLECULAR COLLECTOR SWAB SYSTEM

(76) Inventor: PETER A.K. YONG, TORRANCE, CA (US)

Correspondence Address: WALTER A. HACKLER, Ph.D.;PATENT LAW OFFICE, 2372 S.E. BRISTOL STREET, SUITE B, NEWPORT BEACH, CA, 92660-0755, US

(21) Appl. No.: 11/848,851

(22) Filed: Aug. 31, 2007

Related U.S. Application Data
(60) Provisional application No. 60/842,838, filed on Sep. 6, 2006.

(51) Int. Cl. A61B 10/02

(52) U.S. Cl. 600/572

(57) ABSTRACT

A segmented collector swab system includes a skewer and a plurality of absorbent pads assembled on the skewer in a kebobbed manner.

This application claims priority from U.S. Provisional Patent Application Ser. No. 60/842,838 filed Sep. 6, 2006, which is incorporated herewith in its entirety.

The present invention is generally related to devices for the collection, transport and storage of bio-molecular diagnostic specimens, for example, oral fluids, genomic DNA, buccal cellular DNA or the like thereof for bio-molecular diagnostic testing and forensic genomic identification applications.

With regard to genetic and forensic genomic identification testing, it has long been recognized that the buccal cavity is a convenient location for the collection of saliva DNA and buccal cellular DNA specimen. The buccal cavity of the mouth is anatomically disposed between the inside of the cheek and the gums and teeth. Recent development have shown that DNA sample from saliva or shed buccal cellular tissue is as good qualitatively as DNA from blood samples.

Heretofore, non-blood bio-molecular DNA sampling and assay for parental identification, crime scene investigation, felony profiling and forensic evidence analysis have typically required multi-step actions from the time of collection until analysis at a laboratory. Thus, these procedures, in a high volume lab, require significant time consuming multiple steps involving rapid drying, accurate cutting and effective dispensing and eventual storage of the remaining un-tested absorbent pads or discs.

During the collection process of DNA samples from the buccal cavity, it is not uncommon for the absorbent pad to rub against the buccal cheek mucosal lining to mechanically induce shedding of the cellular tissue to be attracted and absorbed onto the pre-cut absorbent segmented discs.

In order to collect multiple samples, prior art devices have also utilized single large absorbent pads that must be subsequently cut or punched out using expensive and cumbersome machines. This requires additional time consuming procedures and opens the possibility of sample cross contamination which will affect any serious legal outcome of the DNA evidence.

The bio-molecular collection, transport, dispensing and storage system in accordance with the present invention comprises of a fully integrated, multi functioning device. This collector comprises the following;

A device pre-loaded with multi-segmented absorbent discs or pads capable of providing multi samples by using one device that is color-coded or bar coded to insure sample integrity.

The device is capable of dispensing DNA stained discs serially, rapidly and accurately into the specific DNA testing tubes.

The device is a closed, self-contained system to insure hygienic that, more importantly, eliminates sample cross contamination and tampering, and in one embodiment of the present invention, a single device can be utilized for attaching two sets of samples, one for lab testing and another for archival purposes.

Finally, the device is ergonomically designed for simple use.

### SUMMARY OF THE INVENTION

A segmented collector swab system in accordance with the present invention generally includes a skewer and a plurality of absorbent pads, or discs, assembled on the skewer in a kebobbed manner. Each of the pads is separable from the skewer and adjacent pads. The system may include back-to-back swabs to be used to obtain two sets of specimens, one for testing and the other for archival purposes.

More particularly, the system may further comprise a plurality of spacers disposed between adjacent pads on the skewer. In addition, a collar, or sleeve, may be disposed on the skewer in a spaced apart relationship with an end of the skewer and a fixture disposed at the end of the skewer is provided for containing the plurality of pads and a plurality of spaces between the collar, or sleeve, and the fixture end. The fixture is removable allowing the pads to be individually dispensed from the skewer in a serial manner, thus enabling a plurality of specimens, disposed on each of the pads after collection, to be selectively deposited in one or more diagnostic test devices.

In one embodiment, a sleeve is utilized to slide the pads, or discs, from the skewer, alternatively, a fork may be provided for separating and removing each of the pads from the skewer. Each of the pads may include a soft, non-traumatic serrated perimeter for scraping cellular tissue to enhance the gathering of sample from, for example, a buccal mucous lining. To enhance the collection of samples each of the spacers has a diameter less than the diameter of the pad adjacent thereto in order to ensure contact of the pad with tissue.

In another embodiment of the present invention, the collective swab system further includes a handle which supports the skewer with an end of the skewer protruding from the handle.

A plurality of absorbent pads, or discs, are assembled on the skewer protruding end of the kebobbed manner with each pad being separable from the skewer protruding end and adjacent pads.

Further, a dispensing mechanism is provided for withdrawing the skewer protruding end into the handle thereby forcing each pad off the skewer protruding end in a sequential manner whereby the disc is deposited rapidly and safely into the appropriate testing device. The mechanism may be, for example, screw threads disposed on the skewer and engageable screw threads in the handle. In this manner, rotation of the handle will cause the withdrawal of the skewer protruding end into the handle with each of the pads being forced off the skewer as hereinabove noted.

In another embodiment of the present invention, the segmented collector system includes an elongate enclosure having open end along with a skewer slidably disposed within the elongate structure.

A plurality of absorbent pads, or discs, are assembled on the skewer in a kebobbed manner with each pad being separable from the skewer and adjacent pads. More particularly, a skewer moving piston is provided which is disposed within an elongate enclosure and includes an engageable end disposed proximate one end of the elongated enclosure. A plunger handle is provided which is removably engageable with the piston engageable end for enabling manual sliding of the skewer and movement of the pads between a position inside the elongate enclosure and a position exterior another end of the elongate enclosure.

Removable enclosure caps are provided for sealing the elongate enclosure.

More particularly, the plunger handle may comprise a tube storable within the elongate enclosure between the skewer and an inside of the elongate enclosure.

In addition, the system may include a vile including an active agent which is attachable to the enclosure for the introduction of an active agent onto the skewer pads.

A plurality of spacers may be provided and disposed on the skewer between adjacent pads and a collar, or sleeve, may be disposed on the skewer in a spaced apart relationship with an end of the skewer. A fixture may be disposed at the end of the skewer for containing the plurality of pads and the plurality of spacers between the collar and the fixture.

As earlier described, this embodiment also may include a fork for separating and removing each of the pads from the skewer during dispensing or depositing the pads or discs into a test tube.

Alternatively, the system may include a handle for supporting the skewer with an end of the skewer protruding from the handle. In this embodiment, the absorbent pads, or discs, are assembled on the skewer protruding end and a mechanism is provided for withdrawing the skewer protruding an end to the handle thereby forcing each pad off the skewer protruding end.

A method for collection of multiple specimens in accordance with the present invention generally includes exposing skewer kebobbed absorbent pads to a specimen collection site, such as the inner buccal side of the angle of the mouth, and thereafter rubbing the kebobbed pads against the buccal mucous lining to collect and transfer concentrated specimens onto the discs.

Subsequently, the pads are withdrawn from the sampling sites and removed from the skewer individually to provide a multiplicity of specimen containing pads.

### BRIEF DESCRIPTION OF THE DRAWINGS

The present invention may be more readily understood by consideration of the following detailed description when taken in conjunction with the accompanying drawings, in which:

FIG. 1 is a plan view of a segmented collection swab system in accordance with the present invention showing back-to-back detachable swabs each having a handle, skewer, kebobbed pads and a cap;

FIG. 2 is a perspective exploded view showing a skewer and a plurality of absorbent pads assembled on the skewer in a kebobbed manner;

FIG. 3 is an illustration utilizing one part of the two back-to-back detachable collecting system as shown in FIG. 1 for the collection of Genomic DNA samples from crime scene locations, vaginal secretion in sexual assault cases and, buccal cavity (not shown). After sampling with the first part of the collector, the kebobbed discs are capped with a vented cap, and, the same collection procedure is repeated using the second part of the collector device. The two identical parts of the collection system are the disengaged or separated at the mid line joint. Each capped specimen collector system is then transported to the lab and/or archiving facility;

FIG. 4 is an illustration of the dispensing or deposition of absorbent pads from the skewer following collection of specimen thereof and depositing into a test tube, both the skewer and the test tube being identifiable by barcodes;

FIG. 5 is an illustration of the separation of absorbent pads from the skewer using a fork;

FIG. 6 is a cross sectional view of an embodiment with the skewer and kebobs disposed within a vented enclosure along with removable enclosure caps for sealing the enclosure;

FIG. 7 is an illustration showing the coupling of a reagent vial with the enclosure in order to draw reagent into the enclosure to inter-react with the absorbent specimen pads while on the skewer to preserve the specimens thereon when thereafter analyzed in a time sensitive procedure;

FIG. 8 is a cross sectional view of yet another embodiment of the present invention in which a handle includes a tube for insertion into the enclosure between the skewer and the enclosure to provide a compact system;

FIG. 9 is an illustration of the system shown in FIG. 8 with the handle removed from the interior of the enclosure and coupled with a plunger for extending the skewer and pads exterior to the enclosure and withdrawal of the skewer into the enclosure as shown in FIG. 9; and

FIG. 10 is yet another embodiment of the present invention further providing a cork screw mechanism for withdrawing the skewer within the handle supporting the skewer in order to force pads from the skewer as the skewer is withdrawn into the handle by rotation thereof.

### DETAILED DESCRIPTION

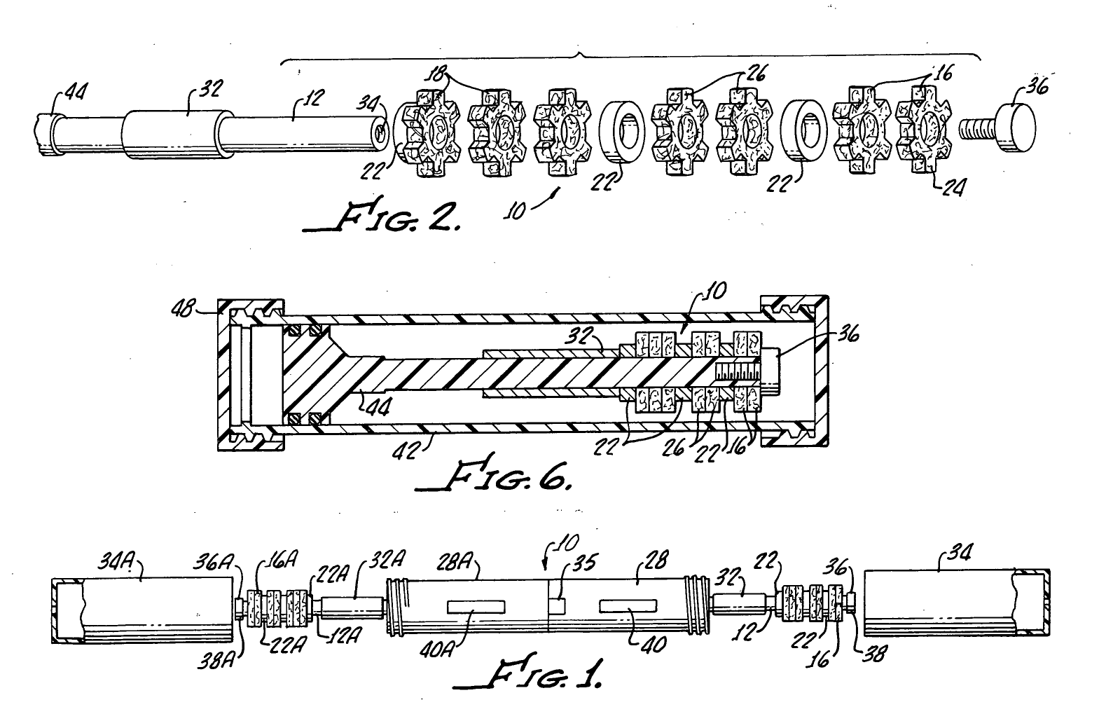

With reference to FIGS. 1 and 2, there is shown a segmented collective swab system 10 in accordance with the present invention which generally includes a skewer 12 and a plurality of absorbent pads, or discs, 16 with only a few being referenced by numbers, the number of pads 16 being any desired number.

Spacers 22 may be disposed between the absorbent pads 16 in order to enhance exposure of the pads 16 to DNA tissue adherence and trapping at a specimen collecting site. This is achieved by providing spacers 22 with diameters less than the diameter of the adjacent pads 16.

In order to enhance cellular shedding and sample concentration, each of the pads 16 may have soft and non-traumatic serrated perimeters 24 (FIG. 2).

The skewer 12 is supported by a handle 28 and a sleeve 32 may be disposed on the skewer 12 for sliding the pads 16 off the skewer 12 as will be hereinafter described in greater detail.

A vented cap 34 may be provided for protection of the pads 16 prior to and after collection of specimens. The cap also serves as a convenient protective transport enclosure for the collector device. The vented perforation at the top of the cap is to enhance rapid disc drying process during the transporting period.

The handle 28 may be removably coupled with a second handle 28A in a back-to-back relationship by a pin 35. Corresponding elements are indicated by the suffix A. This embodiment enables the collection of two sets of specimens, as hereinabove noted, one obtain for testing and another for archival purposes, which is legally required as stored evidence for future court application.

A removable fixture 36 is disposed on the skewer 12 for holding the pads 16 and spacers 22 on the skewer before and during the rubbing collection process.

The fixture 36 may be a separate removable pin lodged in a skewer end 38 or it may be integrally formed in a skewer end 38 for subsequent removal by a clipper or scissors (not shown) to enable disc separation of the pads 16 and spacers 22 from the skewer 12 during dispensing process.

It should be appreciated that the system 10 provides for genomic analysis sampling device which is for forensic genotyping such as bone marrow donor genotyping, veterinary genotyping, paternity identification, immigration profiling, sex offender profiling, as well as for use in natural disasters and battlefield identification.

When used for paternity identification, i.e. clinical genotyping, the pads 16 as well as the handle 28 may be color-coded thus preselecting each of the device for use with a father, mother, and children. A flat surface 40 may be provided for applying a bar code to insure sampling integrity.

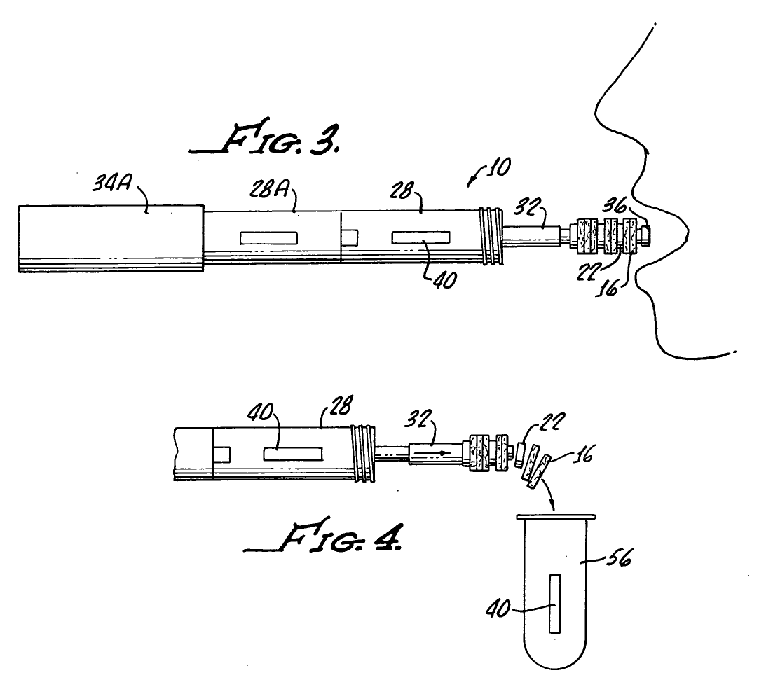

FIG. 3 illustrates the system 10 as it may be inserted into a mouth 52 for obtaining a buccal DNA sample (not shown). FIG. 3 is merely illustrative of one mode of sample collection. The serrated collection pads 16 may be rubbed or stroked within the buccal cavity (not shown) in order to ensure high concentration of shed cells and optimal trapping of DNA specimen on each of the pads 16.

As illustrated in FIG. 4, after collection of specimens, the pads 16 on the skewer may be transported to the lab. At the lab the fixture 36 is removed by severing same or twisting the fixture 36 off the skewer 12 and sliding the sleeve 32 toward the skewer end 38. This mechanism will dispend the disc 16 into a test tube 56.

Alternatively, as illustrated in FIG. 5, a dispensing fork 58 may be utilized to individually separate the pads 16 for deposition in individual test tubes 56. It should be appreciated any number of pads 16 may be removed at one time.

In addition, or in the alternative, to the color-coding hereinabove noted with regard to paternity identification, the handle 28 may be provided with a barcode 62 corresponding to a barcode disposed on the corresponding test tube 56.

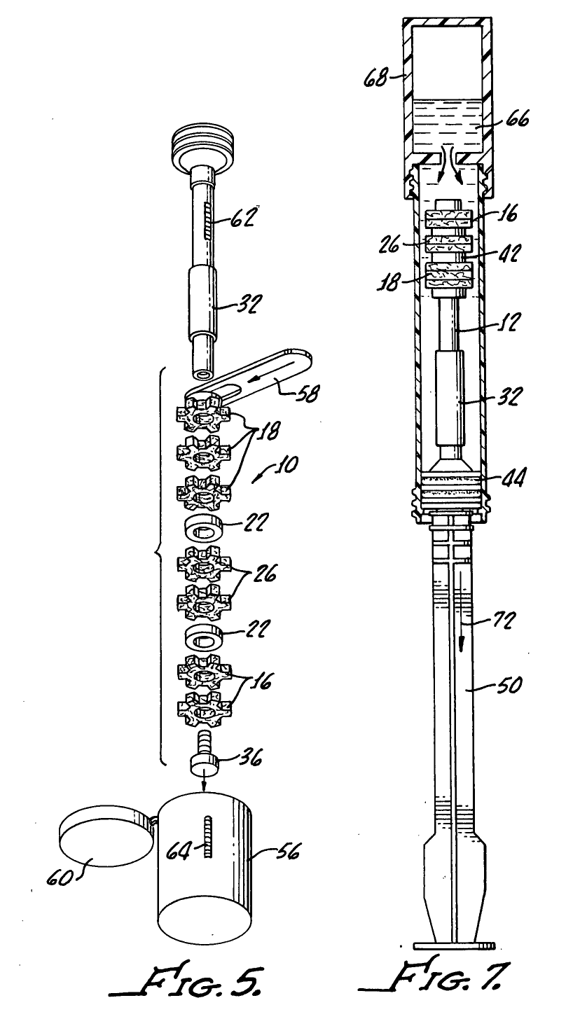

With reference to FIG. 6, an enclosure 42 may be provided which receives the skewer 12 pads 16 and spacers 22 along with a skewer piston 44 and sealed within the elongate enclosure 42 by the enclosure caps 46, 48. This enclosed system is suitable for providing uncontaminated conditions both before use of the system and following the use of the system in collecting a specimen.

In FIG. 7, the plunger handle 50 is removably engageable with the skewer piston 44 and enables manual sliding of the skewer 12 within the enclosure.

If desired, as illustrated in FIG. 7, the sample pads 16 may be treated, preserved or reacted with a reagent 66 from a reagent vial 68 couplable to an end 70 of the enclosure 42 by movement of the piston 44 with a skewer and pads 16 by the plunger handle 50 either by submerging the sample pads 16, 18 into the reagent 16 or drawing a reagent from the coupled reagent vial 68 via the plunger handle 50 as indicated by the arrow 72.

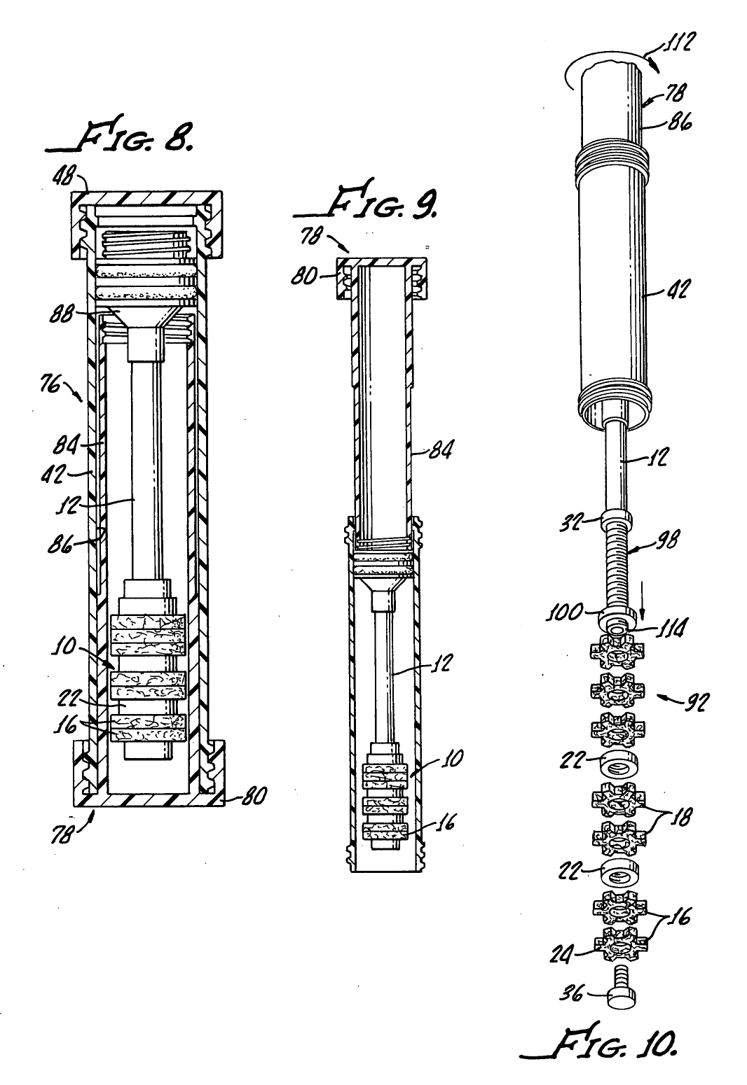

An alternative segmented collector swab system 76 is illustrated in cross section in FIGS. 8 and 9 with identical or substantially similar component indicated by common reference characters set forth in description of system 10.

In FIG. 8, this system 76, a handle 78 includes a cap 80 for sealing the elongate enclosure 42 and also includes a tube 86 storable within the elongate enclosure 42 between the skewer 12 and an inside 86 of the enclosure 42. As illustrated in FIG. 8, this provides for compact storage of the system 76.

In use, as shown in FIG. 9, the tube 86 is removed from the elongate enclosure 42 and coupled to plunger 88 to enable manual sliding the skewer 12 and the assembled pads 16 between a position interior of the enclosure 42 and a position exterior to the enclosure 42 following coupling of the tube 86 to the plunger 88 as shown in FIG. 10.

Another dispensing embodiment system 92 in accordance with the present invention as shown in FIG. 10 with common reference characters indicating identical or substantive similar components as hereinabove described in connection with the system 76. In this embodiment, a handle 94 supports a threaded skewer 98 with an end 114 protruding from the handle 94. Absorbent pads 16 and spacers 22 are assembled on the protruding end 100 in a manner as described in connection with the system 10 shown in FIG. 1.

The system 92 includes a cork and screw mechanism 102 with threads 104 on a skewer 98 and screw threads 106 on an inside 108 of the handle 94 thereby enabling withdrawal of the protruding end 100 into the handle 94 by rotation of the handle 94 as indicated by the arrow 112 in order to force off the pads 16 from the skewer 98.

It should be appreciated any mechanism (not shown) may be utilized to withdraw the skewer 98 into the handle to force the pads 16 of the skewer.

A method in accordance with the present invention includes exposing skewer kebobbed pads to a specimen collection location as illustrated in FIG. 3 and rubbing the pads against the sites to shed, attract and transfer DNA specimens thereon.

Subsequent withdrawal of the skewer with pads 16, 18 thereon the pads are removed from the skewer 12 to provide a multiplicity specimen containing pads 16.

Although there has been hereinabove described a specific segmented collection swab system and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A segmented collector swab system comprising:
a skewer; and
a plurality of absorbent pads or disc assembled on said skewer in a kebobbed manner, each pad being separable from said skewer and adjacent pads.

2. The system according to claim 1 further comprising a plurality of spacers disposed on said skewer between adjacent pads.

3. The system according to claim 2 further comprising a fixture disposed at an end of said skewer for containing the plurality of pads and said plurality of spaces on said skewer.

4. The system according to claim 1 further comprises a dispensing fork for separating and removing each of the pads from said skewer into the testing tube.

5. The system according to claim 1 wherein each pad includes a serrated perimeter to enhance mechanical scrapping or shedding of buccal cells.

6. The system according to claim 2 wherein each pad includes a serrated perimeter for scraping tissue and each spacer has a diameter less than a diameter of each pad.

7. The system according to claim 1 further comprising a dispensing sleeve disposed on said skewer for sliding the pads off the skewer into a test tube during DNA eluting process.

8. A segmented collector swab system comprising:
a handle;
a skewer supported by said handle and having an end protruding from said handle;
a plurality of absorbent pads assembled on the skewer protruding end in a kebobbed manner, each pad being separable from the skewer protruding end and adjacent pads; and
a mechanism for withdrawing the skewer protruding end into the handle and forcing each pad off the skewer protruding end.

9. The system according to claim 8 wherein the mechanism comprises screw threads on said skewer and engageable screw threads in said handle.

10. A segmented collector swab system comprising:
a first handle;
a first skewer supported by said first handle and having an end protruding from said first handle;
a plurality of first absorbent pads assembled on the first skewer protruding end in a kebobbed manner, each first pad being separable from the first skewer protruding end and adjacent first pads;
a second handle;
a second skewer supported by said second handle and having an end protruding from said second handle; and
a plurality of second absorbent pads assembled on the second skewer protruding end in a kebobbed manner, each second pad being separable from the second skewer protruding end and adjacent second pads;
the first and second handles being removably attached to one another in a back-to-back relationship, severability of the handle enabling the first absorbent pads to be used for testing and the second absorbent pads to be used for archival purposes.

11. A segmented collector swab system comprising:
an elongate enclosure having open ends;
a skewer slidably disposed within said elongate enclosure;
a plurality of absorbent pads assembled on said skewer in a kebobbed manner, each pad being separable from said skewer and adjacent pads;
a skewer moving skewer piston, disposed within said elongate enclosure, having an engageable end disposed proximate one end of said elongate enclosure;
a plunger handle, removably engageable with the piston engageable end, for enabling manual sliding of said skewer and the assembled pads between a position inside of said elongate enclosure and a position exterior another end of said elongate enclosure; and
removable enclosure caps for sealing said elongate enclosure.

12. The system according to claim 11 wherein said plunger handle comprises a tube storable within said elongate enclosure between said skewer and an inside of said elongate enclosure.

13. The system according to claim 11 further comprising an active agent containing vial, attachable to the enclosure for introduction of an active agent to the skewered pads.

14. The system according to claim 11 further comprising a plurality of spacers disposed on said skewer between adjacent pads.

15. The system according to claim 14 further comprising a collar disposed on said skewer in a spaced apart relationship with an end of said skewer and a fixture disposed at said end of said skewer for containing the plurality of pads and said plurality of spaces between said collar and said fixture.

16. The system according to claim 11 further comprises a fork for separating and removing each of the pads from said skewer.

17. The system according to claim 11 wherein each pad includes a serrated perimeter for scraping tissue.

18. The system according to claim 14 wherein each pad includes a serrated perimeter for scraping tissue and each spacer has a diameter less than a diameter of each pad.

19. The system according to claim 11 further comprising a handle for supporting said skewer with an end of said skewer protruding from said handle, said absorbent pads being assembled on the skewer protruding end and a mechanism for withdrawing the skewer protruding end into the handle for forcing each pad off the skewer protruding end.

20. The system according to claim 19 wherein the mechanism comprises screw threads on said skewer and engageable screw threads in said handle.

21. A method for collection of multiple specimens, said method comprising:
exposing skewer kebobbed absorbent pads to a specimen location;
rubbing the pads against tissue at the location to transfer specimens thereon;
withdrawal of the pads form the location;
removing the pads from the skewer to provide a multiplicity of specimen containing pads.
